(19) United States
(12) Patent Application Publication
(10) Pub. No.: US 2019/0008699 A1
(43) Pub. Date: Jan. 10, 2019

(54) WEARABLE NAPKIN

(71) Applicant: Will Lanier, Atlanta, GA (US)

(72) Inventors: William Preston Lanier, Atlanta, GA (US); Will Lanier, Atlanta, GA (US); Desiree Lanier, Atlanta, GA (US)

(21) Appl. No.: 15/982,177

(22) Filed: May 17, 2018

Related U.S. Application Data
(63) Continuation-in-part of application No. 29/628,681, filed on Dec. 6, 2017.
(60) Provisional application No. 62/530,415, filed on Jul. 10, 2017.

(51) Int. Cl. A61F 13/47; A41D 19/00; A47G 11/00; A47G 21/16

(52) U.S. Cl. CPC A61F 13/47; A41D 19/00; A41D 2400/52; A47G 21/16; A47G 11/002

(57) ABSTRACT

Disclosed is a wearable napkin configured to be worn on a hand. The wearable napkin may include an upper layer composed of a paper material. Further, an upper contour of the upper layer corresponds to a contour of the hand. Further, the wearable napkin may include a lower layer composed of the paper material attached to the upper layer forming a volume therebetween configured to receive the hand. Further, a lower contour of the lower layer corresponds to the contour of the hand.

### FIELD OF THE INVENTION

The present invention relates generally to hand gloves. In particular, the present invention relates to a napkin glove and a method of manufacturing the napkin glove.

### BACKGROUND OF THE INVENTION

Across the world, people eat various types of food with their bare hands. Such types of food may include snacks (such as chips), pizza, etc. In general, the user's hand may get dirty and messy while eating food with their bare hands.

Furthermore, hands are one of the main tools that humans use all day and are often used to touch various non-sterile surfaces such as keyboards, mobile devices, door knobs etc. Accordingly, the hands may not be totally clean when people eat food their bare hands. Therefore, various kinds of microorganisms may be ingested while eating food with bare hands. This may cause infectious diseases like dysentery, cholera, influenza etc.

Accordingly, there is a need for a solution that may also overcome one or more of the abovementioned problems and/or limitations.

### SUMMARY OF THE INVENTION

This summary is provided to introduce a selection of concepts in a simplified form that are further described below in the Detailed Description. This summary is not intended to identify key features or essential features of the claimed subject matter. Nor is this summary intended to be used to limit the claimed subject matter's scope.

Disclosed is a wearable napkin configured to be worn on a hand is provided. The wearable napkin may include an upper layer composed of a paper material. Further, an upper contour of the upper layer corresponds to a contour of the human hand, the backhand portion in particular. Further, the wearable napkin may include a lower layer composed of the paper material attached to the upper layer, forming a volume therebetween configured to receive the hand of a user. Further, a lower contour of the lower layer corresponds to the contour of the user's hand, the palm portion in particular.

According to some embodiments, a method of manufacturing a wearable napkin configured to be worn on a hand is also provided. The method may include forming an upper layer composed of a paper material. Further, an upper contour of the upper layer corresponds to a contour of the hand. Further, the method may include forming a lower layer composed of the paper material. Further, a lower contour of the lower layer corresponds to the contour of the hand. Further, the method may include attaching the upper layer to the lower layer in order to form a volume therebetween configured to receive the hand.

According to some embodiments, a snack glove may be disclosed. The snack glove may a napkin that is in the shape of a hand that fits on the hand while in use. The snack glove allows users to eat food without getting the hands messy. This protects the hands from chip dust, candy, cookies, cupcakes, ice-cream cones and more. Further, it protects the users from various infectious diseases.

According to further embodiments, the components of the snack glove include two pieces of durable paper towel like paper bonded together with some sort of resin or sewed with a paper thread. The snack glove may be worn on either the right hand or the left hand. Further, the snack glove may be available for kids and adults.

According to some embodiments, the snack glove may be attached to food packages. Accordingly, a user may remove the snack glove from a food package, wear the snack glove on a hand and then consume the snack using the hand with the snack glove. Thereafter, the snack glove may be thrown away, leaving the hand clean.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants.

In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a front view of a wearable napkin configured to be worn on a hand, in accordance with some embodiments.

FIG. 2 is a back view of the wearable napkin, in accordance with some embodiments.

FIG. 3 shows a front view of the wearable napkin with an uneven surface, in accordance with some embodiments.

FIG. 4 is a front view of the wearable napkin with segmented finger portions, in accordance with some embodiments.

FIG. 5 illustrates the wearable napkin with a design, in accordance with some embodiments.

FIG. 6 illustrates the wearable napkin with a design, in accordance with some embodiments.

FIG. 7 is a flowchart of a method of manufacturing the wearable napkin configured to be worn on a hand in accordance with some embodiments.

FIG. 8 illustrates the wearable napkin in use for holding various edible objects, in accordance with some embodiments.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure.

Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of hand gloves, embodiments of the present disclosure are not limited to use only in this context.

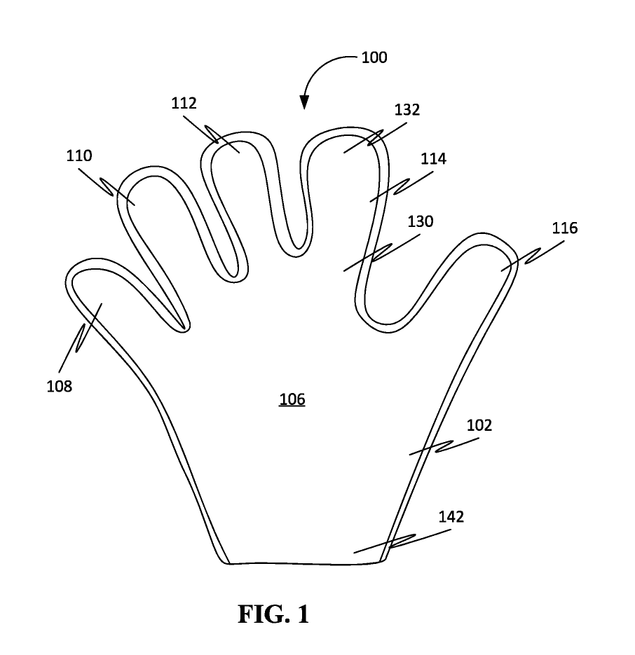

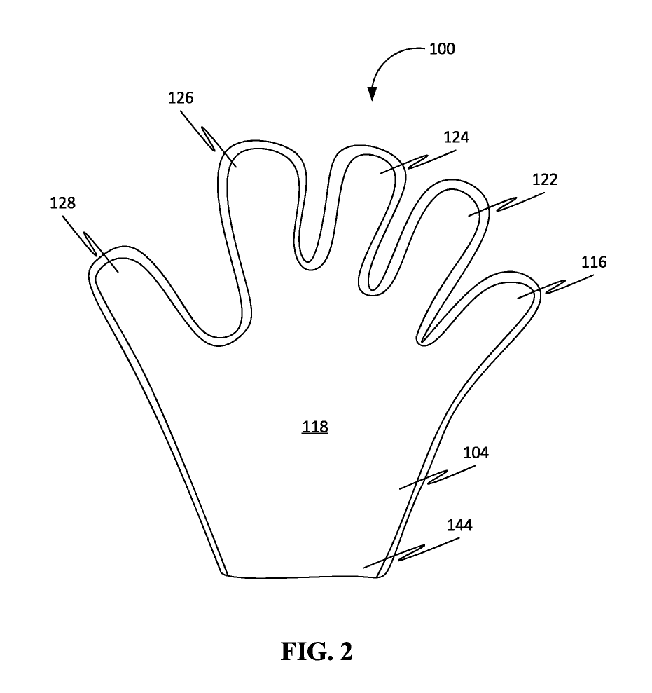

Referring now to figures, FIG. 1 is a front view of a wearable napkin 100 configured to be worn on a hand, in accordance with some embodiments. FIG. 2 is a back view of the wearable napkin 100. The wearable napkin 100 may include an upper layer 102 composed of a paper material. Further, the wearable napkin 100 may include a lower layer 104 composed of the paper material attached to the upper layer 102 forming a volume therebetween configured to receive the hand.

In some embodiments, the paper material may include one or more of a woven paper material and a non-woven paper material.

In some embodiments, the paper material may include one or more of tea-bag paper, parchment paper, pulp paper and tissue paper.

In some embodiments, the paper material may include a food-grade paper material. Therefore, the paper material may not harm users if the users consume bits of paper material while eating food using the wearable napkin 100.

In some embodiments, the upper layer 102 may be attached to the lower layer 104 by sewing the upper layer 102 to the lower layer 104 using a paper thread. In some embodiments, the upper layer 102 may be attached to the lower layer 104 using a resin disposed along a periphery of each of the upper layer 102 and the lower layer 104. Further, the resin may include a food-grade resin. Further, an upper contour of the upper layer 102 corresponds to a contour of the hand. Further, a lower contour of the lower layer 104 corresponds to the contour of the hand.

In some embodiments, each of the upper layer 102 and the lower layer 104 may include a plurality of plys. Further, the plurality of plys may be adhered together using, for example, a bonding agent such as an adhesive. Further, in some embodiments, the plurality of plys may be pealed from each other. Accordingly, for instance, the plurality of plys may be adhered together using a releasable adhesive. As a result, an outer ply of the plurality of ply that may be soiled, due to usage of the wearable napkin 100, may be conveniently peeled off, thus exposing an inner ply of the plurality of plys for further use.

For example, in an instance, a user of the wearable napkin 100 may apply a makeup removal solution to the outer ply. Subsequent to using the wearable napkin 100 for removing makeup, the outer ply may be soiled. Accordingly, the user may be enabled to peel away the outer ply. Alternatively, in some embodiments, one or more plys of the plurality of plys may be impregnated with makeup removal solution and subsequently bonded together. For example, the inner ply may be impregnated by the makeup removal solution, while the outer ply may act as a protective layer to preserve the inner ply. Accordingly, when needed, the user may peel away the outer ply, exposing the inner ply for makeup removal. In some embodiments, the upper layer 102 may include an upper exterior layer and an upper interior layer. Further, the lower layer 104 may include a lower exterior layer and a lower interior layer. Further, each of the upper exterior layer and the lower exterior layer may be composed of paper material characterized by absorbency of one or more of an aqueous solution and an oil-based solution. Further, each of the upper interior layer and the lower interior layer may be composed of paper material characterized by impermeability to at least one the aqueous solution and the oil-based solution. In an another embodiment of the present invention, one from the plurality of plys may be covered in or composed of antimicrobial substances.

In some embodiments, the upper layer 102 may include an upper palm portion 106 and multiple upper finger portions 108-116. Further, the lower layer 104 may include a lower palm portion 118 and multiple lower finger portions 120-128. Further, each upper finger portion in multiple upper finger portions 108-116 may include an upper base part proximal to the upper palm portion 106 and an upper tip part distal to the upper palm portion 106. For example, the upper finger portion 114 may include an upper base part 130 proximal to the upper palm portion 106 and an upper tip part distal 132 to the upper palm portion 106. Similarly, each lower finger portion in the multiple lower finger portions 120-128 may include a lower base part proximal to the lower palm portion and a lower tip part distal to the lower palm portion.

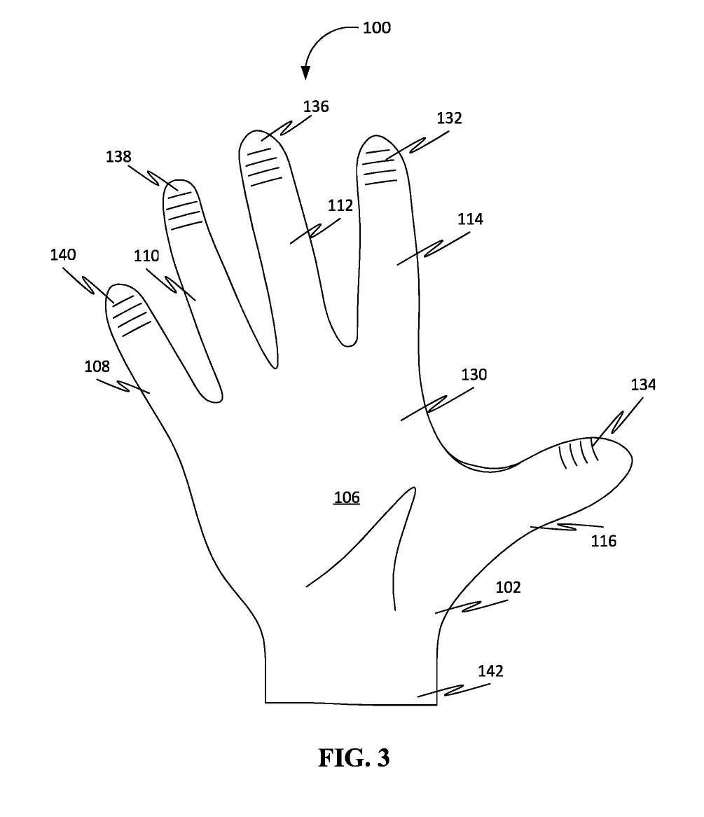

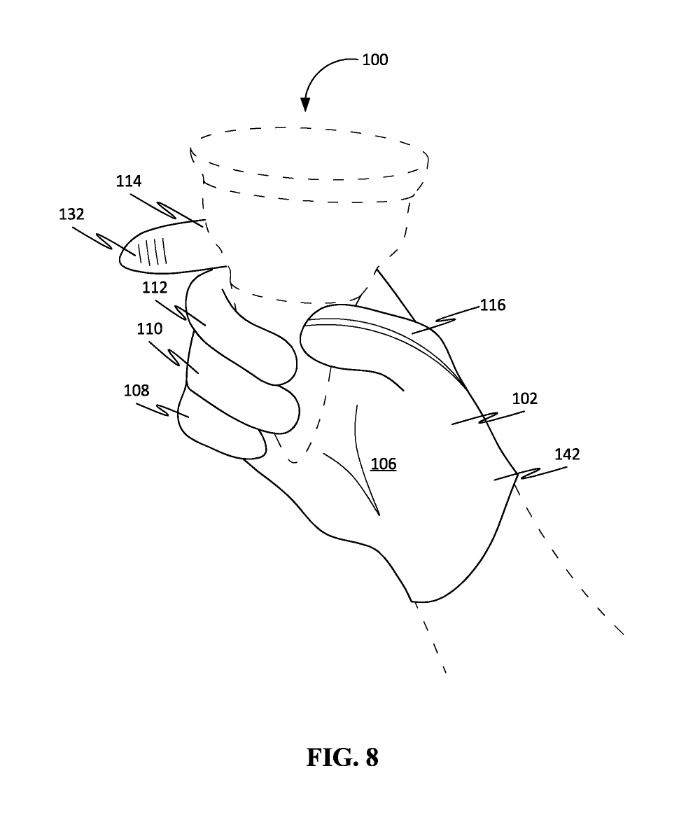

In some embodiments, the lower layer 104 may include an interior side and an exterior side. Further, at least a portion of the exterior side may include an uneven surface configured to facilitate grip. As shown in FIG. 3 and FIG. 8, a portion of the exterior side corresponding to the lower tip part may include an uneven surface configured to facilitate grip. Accordingly, lower tip parts 132-140 may include an uneven surface.

In further embodiments, each of the upper layer 102 and the lower layer 104 may include an interior side and an exterior side. Further, a portion of the exterior side corresponding to the lower tip part and the upper tip part may include an uneven surface configured to facilitate grip.

In further embodiments, the exterior side may include a coating of a hydrophobic material. Further, the interior side may include a disinfectant or antimicrobial coating.

In some embodiments, the wearable napkin 100 may further include a fastening mechanism. Further, the fastening mechanism may be configured to fasten the wearable napkin 100 to a user's hand. Further, the upper layer 102 may include the upper palm portion 106, multiple upper finger portions 108-116 and an upper wrist portion 142. Further, the lower layer 104 may include the lower palm portion 118, the multiple lower finger portions 120-128 and a lower wrist portion 144. Further, the fastening mechanism may be attached to one or more of the upper wrist portion 142 and the lower wrist portion 144.

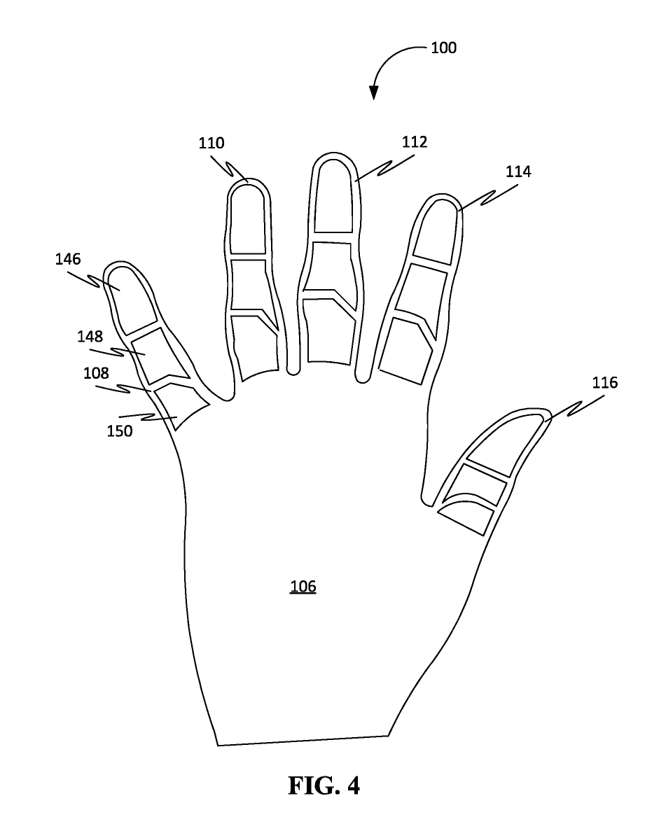

FIG. 4 is a front view of the wearable napkin 100 with segmented finger portions, in accordance with some embodiments. The upper layer 102 may include an upper palm portion 106 and multiple upper finger portions 108-116. Further, each upper finger portion may be segmented into three parts. The three parts may correspond to the three phalanges (bones) in the human hand. The three phalanges may include a distal phalanx, a middle phalanx, and a proximal phalanx. For example, the upper finger portion 108 may be split into three parts 146-150. The segmented finger portions may allow the user to easily move and bend the fingers while using the wearable napkin 100.

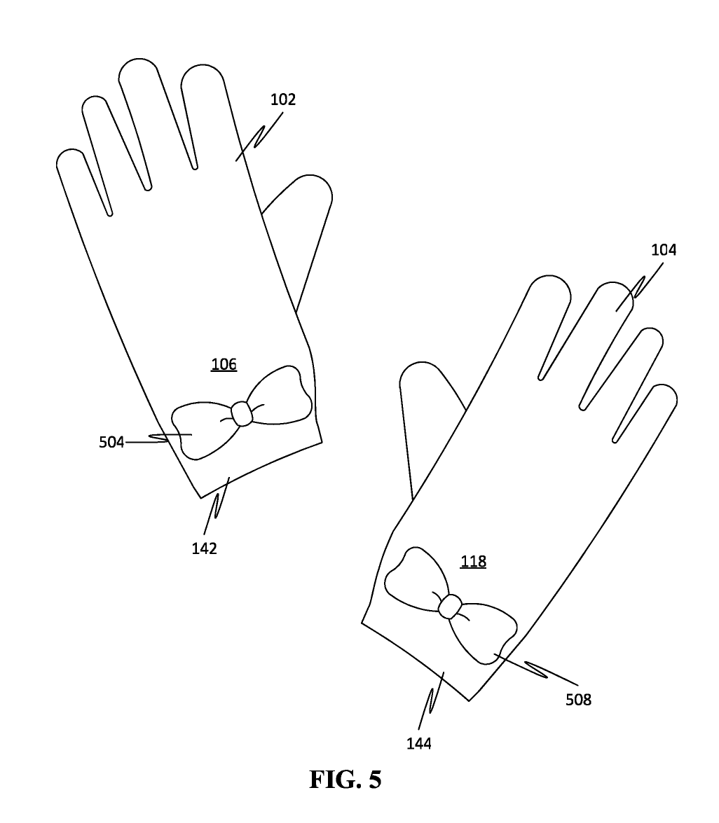

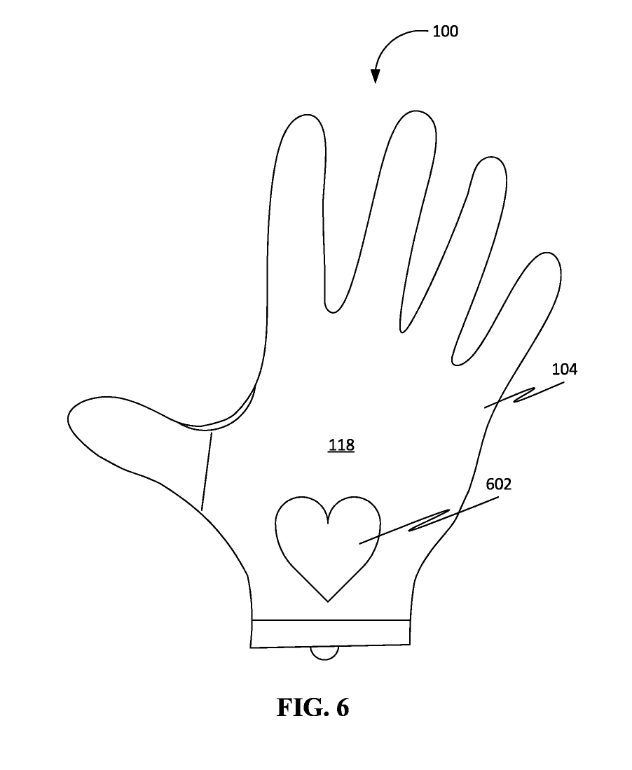

In some embodiments, the wearable napkin 100 may also include one or more design features. FIG. 5 illustrates the wearable napkin 100 with a design, in accordance with some embodiments. The upper layer 102 may include artwork on the upper palm portion 106 and a bow 504 on the upper wrist portion 142. Similarly, the lower layer 104 may include artwork on the lower palm portion 118 and a bow 508 on the lower wrist portion 144. FIG. 6 illustrates a back view of the wearable napkin 100 with a heart-shaped imprint 602 near the lower palm portion 118, in accordance with some embodiments.

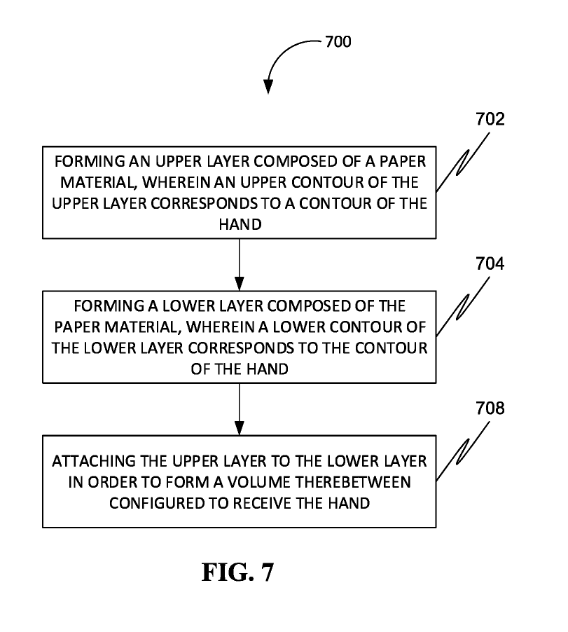

FIG. 7 is a flowchart of a method 700 of manufacturing the wearable napkin 100 configured to be worn on a hand in accordance with some embodiments. At 702, the method 700 may include forming the upper layer 102 composed of a paper material. Further, an upper contour of the upper layer 102 corresponds to a contour of the hand.

Further, at 704, the method 700 may include forming the lower layer 104 composed of the paper material. Further, a lower contour of the lower layer 104 corresponds to the contour of the hand.

Further, at 706, the method 700 may include attaching the upper layer 102 to the lower layer 104 in order to form a volume therebetween configured to receive the hand.

In some embodiments, the attaching may include sewing the upper layer 102 to the lower layer 104 using a paper thread.

In some embodiments, the attaching may include bonding the upper layer 102 to the lower layer 104 using a resin disposed along a periphery of each of the upper layer 102 and the lower layer 104.

In some embodiments, the upper layer 102 may include the upper palm portion 106 and multiple upper finger portions 108-116. Further, the lower layer 104 may include the lower palm portion 118 and multiple lower finger portions 120-128. Further, each upper finger portion may include an upper base part proximal to the upper palm portion 106 and an upper tip part distal to the upper palm portion 106. Further, each lower finger portion may include a lower base part proximal to the lower palm portion and a lower tip part distal to the lower palm portion.

Further, each of the upper layer 102 and the lower layer 104 may include an interior side and an exterior side. Further, the method 700 further may include forming an uneven surface on a portion of the exterior side corresponding to each of the lower tip part and the upper tip part in order to facilitate grip. For example, as shown in FIG. 3, lower tip parts 132-140 may include an uneven surface.

The present invention may be utilized for a multitude of functions including, but not limited to, detailing cars, detailing boats, housekeeping, household chores, gardening, fishing, removal of makeup, painting, applying or removing finish from furniture, computer parts/screen cleaning, preparing food, handling and installing glass, grocery handling and shopping, firearm cleaning, lightbulb handling and changing, and maintenance and building of a watch. As mentioned before, the present invention is also used for snacking to ensure that the user's hands stay clean and for clean up to significantly easier. Additionally, the present invention may also be used for arts and crafts for kids and adults as it provides a disposable protection layer for hands. Furthermore, the present invention may also include antimicrobial properties in order to allow a user to sterilize and clean surfaces.

Although the invention has been explained in relation to its preferred embodiment, it is to be understood that many other possible modifications and variations can be made without departing from the spirit and scope of the invention.

What is claimed is:

1. A wearable napkin configured to be worn on a hand, the wearable napkin comprising:
an upper layer composed of a paper material, wherein an upper contour of the upper layer corresponds to a contour of the hand; and
a lower layer composed of the paper material attached to the upper layer forming a volume therebetween configured to receive the hand, wherein a lower contour of the lower layer corresponds to the contour of the hand.

2. The wearable napkin of claim 1, wherein the paper material comprises at least one of a woven paper material and a non-woven paper material.

3. The wearable napkin of claim 1, wherein the paper material comprises at least one of tea-bag paper, parchment paper, pulp paper and tissue paper.

4. The wearable napkin of claim 1, wherein each of the upper layer and the lower layer comprises a plurality of plys.

5. The wearable napkin of claim 1, wherein the upper layer comprises an upper exterior layer and an upper interior layer, wherein the lower layer comprises a lower exterior layer and a lower interior layer, wherein each of the upper exterior layer and the lower exterior layer is composed of paper material characterized by absorbency of at least one of an aqueous solution and an oil-based solution, wherein each of the upper interior layer and the lower interior layer is composed of paper material characterized by impermeability to at least one the aqueous solution and the oil-based solution.

6. The wearable napkin of claim 1, wherein the lower layer comprises an interior side and an exterior side, wherein at least a portion of the exterior side comprises an uneven surface configured to facilitate grip.

7. The wearable napkin of claim 1, wherein the upper layer comprises an upper palm portion and a plurality of upper finger portions, wherein the lower layer comprises a lower palm portion and a plurality of lower finger portions, wherein each upper finger portion comprises an upper base part proximal to the upper palm portion and an upper tip part distal to the upper palm portion, wherein each lower finger portion comprises a lower base part proximal to the lower palm portion and a lower tip part distal to the lower palm portion.

8. The wearable napkin of claim 7, wherein the lower layer comprises an interior side and an exterior side, wherein a portion of the exterior side corresponding to the lower tip part comprises an uneven surface configured to facilitate grip.

9. The wearable napkin of claim 7, wherein each of the upper layer and the lower layer comprises an interior side and an exterior side, wherein a portion of the exterior side corresponding to the lower tip part and the upper tip part comprises an uneven surface configured to facilitate grip.

10. The wearable napkin of claim 1 further comprising a fastening mechanism, wherein the fastening mechanism is configured to fasten the wearable napkin to the hand, wherein the upper layer comprises an upper palm portion, a plurality of upper finger portions and an upper wrist portion, wherein the lower layer comprises a lower palm portion, a plurality of lower finger portions and a lower wrist portion, wherein the fastening mechanism is attached to at least one of the upper wrist portion and the lower wrist portion.

11. The wearable napkin of claim 1, wherein the upper layer is attached to the lower layer by sewing the upper layer to the lower layer using a paper thread.

12. The wearable napkin of claim 1, wherein the upper layer is attached to the lower layer using a resin disposed along a periphery of each of the upper layer and the lower layer.

13. The wearable napkin of claim 11, wherein the resin comprises a food-grade resin.

14. The wearable napkin of claim 1, wherein the paper material comprises a food-grade paper material.

15. The wearable napkin of claim 1, wherein each of the upper layer and the lower layer comprises an interior side and an exterior side, wherein the exterior side comprises a coating of a hydrophobic material.

16. The wearable napkin of claim 1, wherein each of the upper layer and the lower layer comprises an interior side and an exterior side, wherein the interior side comprises a coating of a disinfectant.

17. A method of manufacturing a wearable napkin configured to be worn on a hand, the method comprising:
forming an upper layer composed of a paper material, wherein an upper contour of the upper layer corresponds to a contour of the hand;
forming a lower layer composed of the paper material, wherein a lower contour of the lower layer corresponds to the contour of the hand; and
attaching the upper layer to the lower layer in order to form a volume therebetween configured to receive the hand.

18. The method of claim 17, wherein the upper layer comprises an upper palm portion and a plurality of upper finger portions, wherein the lower layer comprises a lower palm portion and a plurality of lower finger portions, wherein each upper finger portion comprises an upper base part proximal to the upper palm portion and an upper tip part distal to the upper palm portion, wherein each lower finger portion comprises a lower base part proximal to the lower palm portion and a lower tip part distal to the lower palm portion, wherein each of the upper layer and the lower layer comprises an interior side and an exterior side, wherein the method further comprises forming an uneven surface on a portion of the exterior side corresponding to each of the lower tip part and the upper tip part in order to facilitate grip.

19. The wearable napkin of claim 17, wherein the attaching comprises sewing the upper layer to the lower layer using a paper thread.

20. The wearable napkin of claim 17, wherein the attaching comprises bonding the upper layer to the lower layer using a resin disposed along a periphery of each of the upper layer and the lower layer.
